(19) United States
(12) Patent Application Publication
(10) Pub. No.: US 2017/0113086 A1
(43) Pub. Date: Apr. 27, 2017

(54) METHODS AND SYSTEMS FOR NEUTRALIZATION OF HYDRAZINE

(71) Applicant: Hydrazine Neutralizing Solutions Inc., Marietta, GA (US)

(72) Inventors: Rodolfo Antonio Martinez, Santa Fe, NM (US); David Rembert Glass, Santa Fe, NM (US); Merritt Clemens Helvenston, Albuquerque, NM (US)

(73) Assignee: Hydrazine Neutralizing Solutions Inc.

(21) Appl. No.: 15/336,052

(22) Filed: Oct. 27, 2016

Related U.S. Application Data
(60) Provisional application No. 62/247,003, filed on Oct. 27, 2015.

(51) Int. Cl. A62D 3/37; C07C 251/76

(52) U.S. Cl. CPC A62D 3/37; C07C 251/76; A62D 2101/26

(57) ABSTRACT

Methods of and systems for remediating hydrazine spills, solutions, and hydrazine-contaminated objects including areas thereof comprise reacting 1,1-Dimethylhydrazine with α-ketoacids and adding a reducing agent to the reaction of 1,1-Dimethylhydrazine with said α-ketoacids.

### CROSS REFERENCE TO RELATED PATENT APPLICATIONS

This patent application claims the priority and benefit under 35 U.S.C. §119(e) of U.S. Provisional Patent Application Ser. No. 62/247,003, filed October 27, 2015, entitled “METHODS AND SYSTEMS FOR NEUTRALIZATION OF HYDRAZINE.” U.S. Provisional Patent Application Ser. No. 62/247,003 is herein incorporated by reference in its entirety.

### TECHNICAL FIELD

Embodiments disclosed herein are generally related to decontamination and remediation methods and systems. Embodiments are also related to hydrazine (Hz) and hydrazine-based compounds such as 1,1-dimethylhydrazine (UDMH), and the remediation and/or neutralization of such compounds. The embodiments are also related to methods and systems for remediating hydrazine from hydrazine-contaminated objects, including hydrazine contaminated areas thereof.

### BACKGROUND

An environmentally conscious remediation process for the emergency treatment/disposal of hydrazine fuel spills is needed. The highly toxic hydrazine family of fuels is utilized as rocket propellants in virtually all U.S. space programs including both launch vehicles and satellite propulsion systems. Since large volumes of these fuels are annually shipped all over the United States, accidental major spills of these propellants could potentially occur near populated communities during transport over the nation's highways. In addition, smaller spills could also occur during launch operations and storage at the user facilities.

Current techniques for neutralization of Hydrazine fuel include incineration, citric acid/glycolic acid, chemical oxidation, hypochlorite, hydrogen peroxide, hypochlorite and UV light, chemical reduction, biodegradation, and chemical conversion to an environmentally safe compound.

Each of these methods includes significant downsides. For example, incineration requires the hydrazine waste stream to be diluted to low concentration for safe disposal. Disposal by this method requires moving the still hazardous hydrazine to an incineration site. In the case of an inadvertent release, the generator is liable for any damages to personnel, the general public, and the environment. It is difficult to project a dollar amount on these potential punitive damages, but from a cost analysis perspective, the possibility must be weighed.

In using Citric Acid/Glycolic Acid, the reaction with hydrazines forms an ammonium salt complex. This is an entrapment method rather than a destruction method. Also, changes in pH release the hydrazine in cases of a spill that is treated by this method.

Chemical oxidation is another option. Sodium hypochlorite (NaOCl) and Calcium hypochlorite (Ca(OCl)₂) are used in this method for neutralization of hydrazine. However, samples must be diluted to less than 3% hydrazine. In the case of monomethylhydrazine (MMH) and unsymmetrical dimethylhydrazine (UDMH), carcinogenic N-nitroso compounds, alkylchlorides, and/or other mutagenic species are produced. The reaction of 3% hydrazine allows for complete decomposition of hydrazine to nitrogen gas, sodium, or calcium chloride and water at pH 4 by hypochlorite oxidation. Even at 3% the reaction is extremely exothermic and dangerous. The complete process also requires additional chemicals to neutralize the hypochlorite.

Similarly, oxidation with hydrogen peroxide yields nitrogen gas and water and neutralization must be done on dilute solutions and is undesirably slow in the absence of metal catalysts.

Chemical oxidation and UV light present another option for remediation. Chlorinolysis at pH 5 with simultaneous ultraviolet (UV) illumination is effective at destroying all types of propellant hydrazines in contaminated water. This process requires UV lamp power input and sodium thiosulfate to remove excess chlorine. Such processes have not been implemented in practice because they are prohibitively expensive.

Other options such as chemical reduction via Raney Nickel, Biodegradation, and other chemical conversion techniques are impractical because of safety hazards, toxicity complications, and practicality issues.

More effective methods and systems are needed for remediating hydrazine from contaminated objects, solutions, and areas. The present inventors recognize this need and have invented methods of, and systems for, hydrazine remediation.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, dawns, drawings, and abstract as a whole.

It is therefore, one aspect of the disclosed embodiments to provide methods and systems for hydrazine remediation.

It is another aspect of the disclosed embodiments to provide a method and system for decontamination and remediation of hydrazine spills.

It is an additional aspect of the disclosed embodiments to provide an enhanced method and system for decontamination and remediation of hydrazine and hydrazine-based compounds spills.

The aforementioned aspects and other objectives and advantages can now be achieved as described herein. In the embodiments disclosed herein is a method for neutralizing the harmful effects of hydrazine comprises reacting 1,1-Dimethylhydrazine with α-ketoacids and adding a reducing agent to the reaction of 1,1-Dimethylhydrazine with the α-ketoacids. In an embodiment, the α-ketoacids comprise any salt. The α-ketoacids may comprise at least one of 2-ketoglutaric acid (2KG), sodium 2-ketoglutaric acid (Na2KG), and disodium 2-ketoglutaric acid (Na₂2KG). Adding a reducing agent to the reaction of 1,1-Dimethylhydrazine with the α-ketoacids produces 2-(2,2-dimethylhydrazinylidene)-pentanedioic acid. The reducing agent can comprise at least one of boron and sodium borohydride.

A system for remediating areas contaminated with hydrazine can include a cleaning solution comprising a 2-ketoglutaric acid, a reducing agent for reducing the reaction of 2KG and hydrazine, and a rinsing mechanism for rinsing equipment and/or ground surfaces contaminated with a hydrazine group compound with the cleaning solution. The system may further comprise decontaminating equipment including an aqueous solution managed by, an application mechanism and comprising 2-ketoglutaric acid and an application mechanism for enabling a user in the application of the aqueous solution to equipment and/or ground surfaces accidentally contaminated with a hydrazine group compound, wherein the hydrazine group compound is converted to a stable organic compound as a result of a reaction between the aqueous solution comprising the 2-ketoglutaric acid and the hydrazine group compound and then reduced in situ by a reducing agent such as boron.

### BRIEF DESCRIPTION OF THE FIGURES

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 depicts a schematic diagram depicting a reaction for the remediation of hydrazine in accordance with the disclosed embodiments;

FIG. 2 depicts a reaction of 2-ketoglutaric acid and hydrazine in accordance with the disclosed embodiments;

FIG. 3 depicts a chart of an NMR spectrum, in accordance with an example embodiment;

FIG. 4 depicts a chart of an NMR spectrum, in accordance with an example embodiment;

FIG. 5 depicts a chart of an NMR spectrum, in accordance with an example embodiment;

FIG. 8 depicts a high-level flow chart of operations illustrating logical operational steps, which can be implemented in accordance with an example embodiment; and

FIG. 7 depicts a chart of an NMR spectrum, in accordance with an example embodiment.

### DETAILED DESCRIPTION

The particular values and configurations discussed in these non-limiting examples can be varied and are cited merely to illustrate at least one embodiment and are not intended to limit the scope thereof.

The embodiments will now be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The inventors have previously worked in the present field and have developed different methods and systems for hydrazine remediation. U.S. Pat. No. 7,074,959, titled “Methods and Systems for Hydrazine Remediation,” filed Jul. 11, 2006 describes one such related method developed by the present inventors. U.S. Pat. No. 7,074,959, titled “Methods and Systems for Hydrazine Remediation,” is herein incorporated by reference in its entirety.

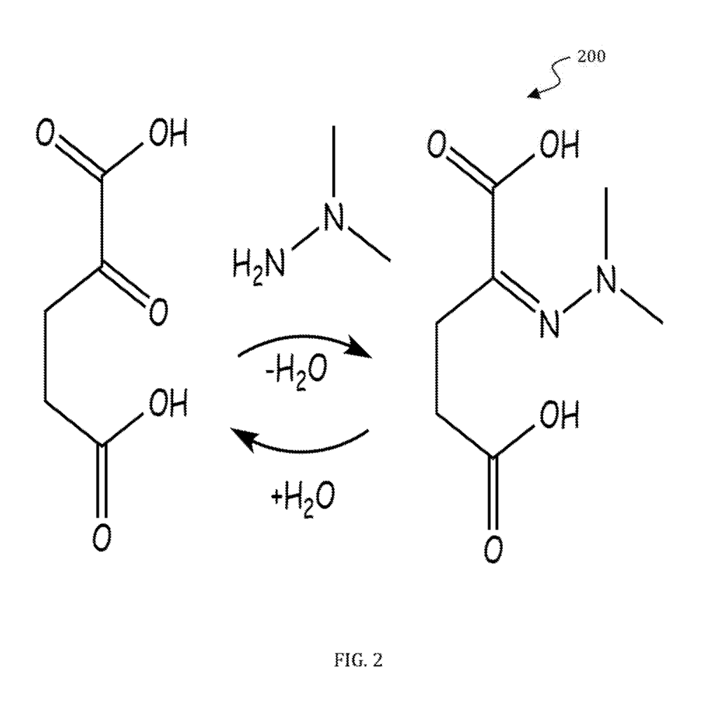

Hydrogenation may not be a practical solution in the field because the reaction of unsymmetrical dimethylhydrazine (UDMH) with 2-ketoglutaric acid (2 KG) is not permanently bound to the resulting product. This is due to the fact that the resulting product can react with the solvent water (H₂O) to easily reverse the reaction depending on the pH and temperature. This can then liberate some measure of UDMH. A reaction 200 of 2 KG with 1,1-Dimethylhydrazine is illustrated in FIG. 2. Note that the figure shows the potential for the reverse reaction described above.

Accordingly, in the embodiments disclosed herein, 2-ketoglutaric acid can be employed to react with hydrazine in a safe and quantitative method for the neutralization of the hydrazine compound (e.g., monomethylhydrazine (MMH) or UDMH). The products of this neutralization are environmentally friendly. Treatment with this method can safely be used for concentrations from 100% to ppb levels. The resulting products from this method may he safe for disposal at common wastewater treatment facilities.

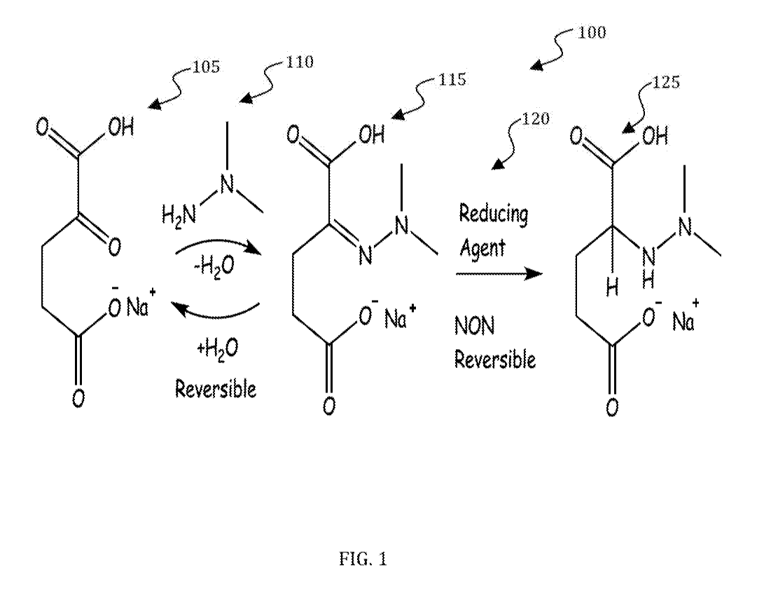

FIG. 1 illustrates a schematic diagram 100 depicting a reaction between 2 KG 105 and a hydrazine complex 110. The reaction between UDMH and 2 KG can be used to create a product 115 as illustrated in FIG. 1. The product 115 from the reaction of UDMH 110 and 2 KG 105 can be converted to a stable nonvolatile organic compound 125 by in situ reduction via a reducing agent 120.

In the diagram illustrated in FIG. 1, the remediation method includes several steps. In first step, the reaction of UDMH 110 and alpha-ketoglutaric acid sodium salt 105 forms 2-(2,2-dimethylhydrazinylidene) pentanedioic acid sodium salt 115.

Next, the product 115 from the reaction of UDMH 110 and 2 KG 105 can be used to bound UDMH in a stable nonvolatile organic compound by in situ reduction. Borohydride can be used as reducing agent The addition of a reducing agent 120 (such as NaBH₄) to the reaction allows for the complete reduction of the 2-(2,2-dimethylhydrazinylidene)-pentanedioic acid sodium salt. It should be understood that other environmentally friendly reducing agents can be used for the reduction including, for example, an aqueous sodium borohydride. The UDMH is thus permanently bound as a new organic molecule. The molecule is solid, nonvolatile, and much safer than unbound UDMH.

In one specific example, when neat UDMH is added to a 1M solution of alpha-ketoglutaric acid sodium salt, the reaction temperature goes up by 6° C. The product 2-(2,2-dimethylhydrazinylidene)-pentanedioic acid sodium salt is formed in 20 minutes. The quantitative product formation can be verified by ¹³C-Nuclear Magnetic Resonance Spectroscopy (NMR). When additional UDMH is added to the reaction above, and there is an excess of UDMH, it can be easily detected by NMR as well.

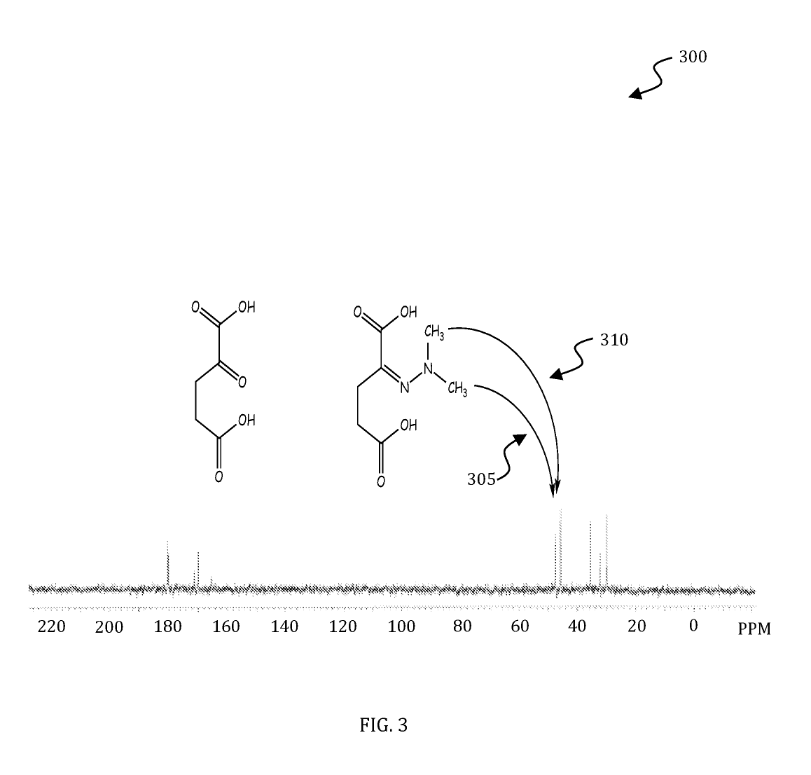

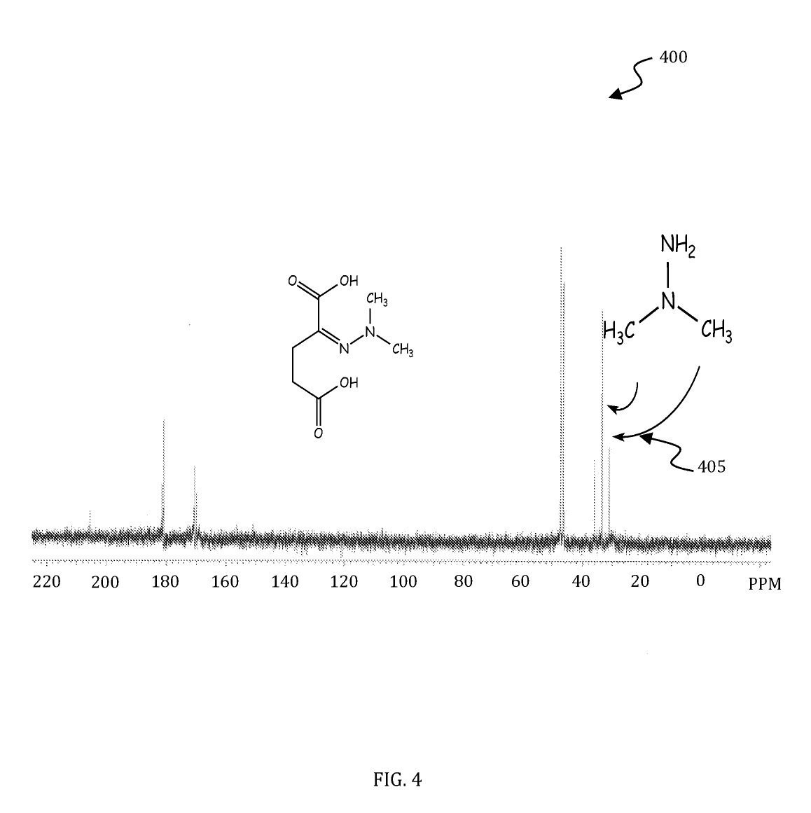

In an exemplary reaction procedure in accordance with the disclosed embodiments, UDMH (for example, 0.5 mL, 0.0066 moles, 1 eq. 99%) is added to an aqueous solution of alpha-ketoglutaric acid sodium salt (5 mL, 0.0088 moles, 1.3 eq) in one rapid addition. The ambient temperature of the solution increases by 6° C. After 30 minutes, the temperature of the solution is back to ambient. After the reaction is allowed to stir for 20 minutes, an NMR of the reaction indicates that all of the UDMH has reacted to form 2-(2,2-dimethylhydrazinylidene)-pentanedioic acid sodium salt as indicated by peak 305 and peak 310 (45 and 47 ppm respectively, (CH₃)₂NN)) as shown in chart 300 of FIG. 3. This pattern indicates that the methyl groups on UDMH are no longer equivalent as expected for 2-(2,2-dimethylhydrazinylidene)-pentanedioic acid sodium salt. In order to prove that all of the UDMH has formed the expected product, additional UDMH (0.5 mL, 0.00663 moles, 1 eq. 99%) is added. The unreacted UDMH appears in an NMR (illustrated in chart 400 of FIG. 4) as a singlet 405 (33 ppm, (CH₃)₂NN). The appearance of unreacted UDMH is pH dependent, occurring in a ¹³C-NMR chemical shift range from 49 ppm to 33 ppm.

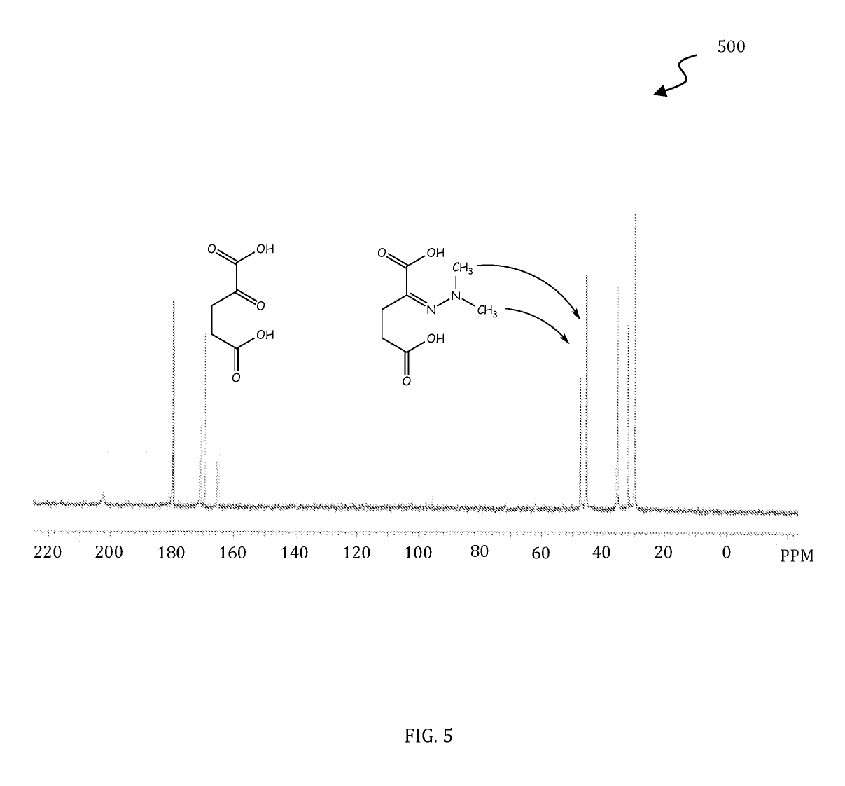

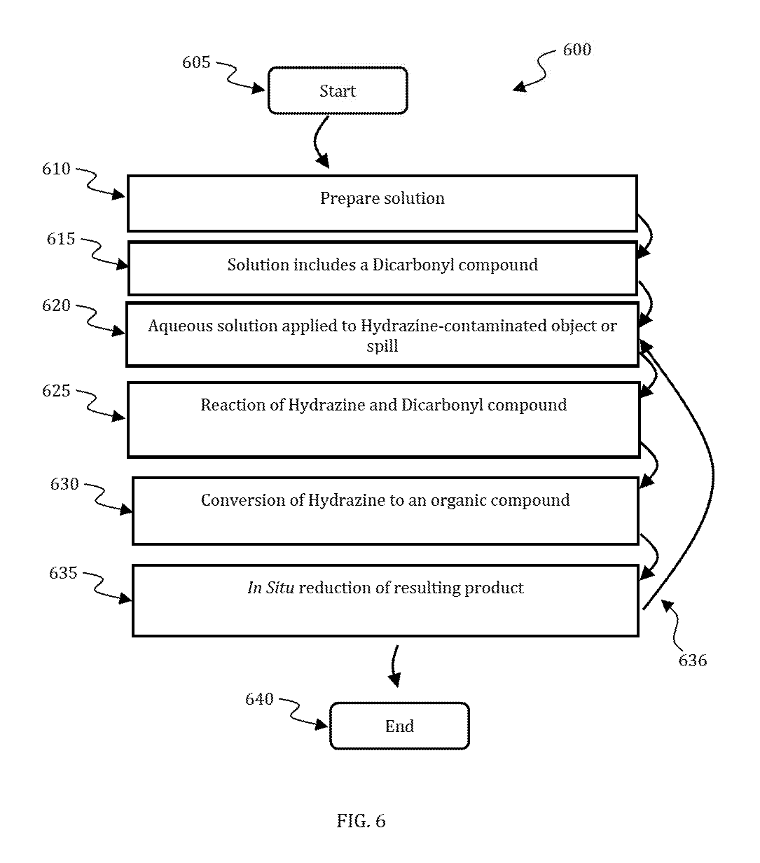

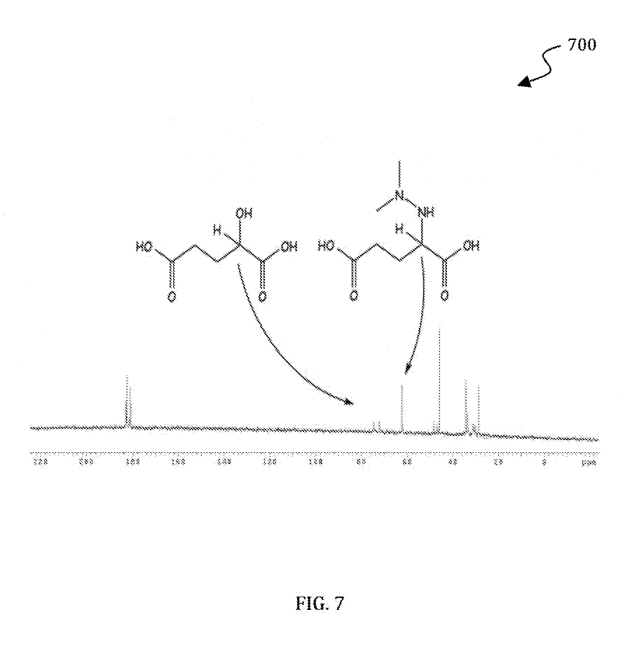

The addition of another 1.3 equivalents of solid alpha-ketoglutaric acid sodium salt completely reacts with UDMH. After an additional 20 minutes, the NMR spectrum, shown in chart 500 of FIG. 5, indicates that the reaction is complete. Evaporation of the reaction using a rotary evaporator yields a foamy material that shows only the expected product 2-(2,2-dimethyl hydrazinylidene)-pentanedioic acid sodium salt and the excess alpha-ketoglutaric acid sodium salt.

The reduction of 2-(2,2-dimethylhydrazinylidene)-pentanedioic acid sodium salt is reduced in situ by the addition of an aqueous sodium borohydride (0.3 gram, 0.008 moles) to give 2-(2,2-dimethylhydrazinyl)-pentanedioic acid plus sodium borate as illustrated in the NMR spectrum in chart 700 of FIG. 7. The addition of alpha-ketoglutarate sodium salt followed by the addition of aqueous sodium borohydride as described above may be repeated in some embodiments. For example, in an exemplary embodiment, the addition of alpha-ketoglutarate sodium salt followed by the addition of aqueous sodium borohydride can be repeated in the manner described above a minimum of 3 times, in order to completely consume the UDMH.

FIG. 6 illustrates a high-level flow chart 600 of steps for the remediation of hydrazine in accordance with an embodiment. The method begins at block 605. As illustrated at block 610, a solution can be prepared that includes, as indicated at block 615, a dicarbonyl-compound. Such a solution can be prepared in the form of a cleaning solution. The solution can be an aqueous solution or a non-aqueous solution. An example of a dicarbonyl-compound that can be utilized in accordance with the methods and systems of the present invention is a keto-acid, such as a 2-ketoglutaric acid. Once the solution has been prepared, it can be provided for utilization in hydrazine remediation.

As indicated next at block 620, the solution can be applied to an object contaminated with a hydrazine group compound. Application can occur, for example, through a rinsing of the hydrazine contaminated object with the solution that includes the dicarbonyl-compound. Examples of hydrazine group compounds that may contaminate an object or area thereof can include hydrazine, MMH, or UDMH.

It should be appreciated by those skilled in the art that the application of the dicarbonyl compound solution to the hydrazine contaminated object and/or area (e.g., a hydrazine spill) thereof can take place following an initial attempt at a physical removal of the hydrazine group compound from the object or area thereof. Wiping, sweeping, scraping, blowing, vacuuming, rinsing with water. and steam cleaning are all examples of physical methods for the removal of contaminants. Such physical removal techniques can thus occur in tandem with the application of the dicarbonyl-compound solution described herein.

A reaction can then occur, as illustrated at block 625 between the hydrazine group compound and the dicarbonyl-compound. As a result of this reaction, the hydrazine group compound is converted to a stable organic compound, as indicated at block 630.

An in situ reduction of the resulting compound with a reducing agent can then be performed as illustrated at block 635, in order to remediate the hydrazine group compound from the object. Due to the reversibility that occurs in block 625 as shown in FIG. I and FIG. 2, block 620 through block 635 can be repeated one or more times as indicated by arrow 636. In an exemplary embodiment, block 620 through block 635 can be repeated a minimum of 3 times for the complete remediation of UDMH. The method ends at block 640.

Accordingly, FIG. 6 illustrates a method that allows for the use of a solution of a dicarbonyl-compound (e.g., 2-ketoglutaric acid) and an in situ reduction for the treatment of hydrazine waste. Such a conversion can occur after application of the dicarbonyl-compound solution through the use of solubilization and dilution processes, among others.

In summary, via the methods disclosed herein, UDMH can be permanently bound as a new organic molecule. The compound can be a solid, is nonvolatile, and is much safer than UDMH. Common wastewater treatment systems and soil bacteria will easily biodegrade the 2-(2,2-dimethylhydrazinyl)-pentanedioic acid. The resulting 2-(2,2-dimethylhydrazinyl)-pentanedioic acid, will have a much lower toxicity rating than that of UDMH and thus disposal of this product will be less expensive. An exemplary reducing agent, Boron, is used extensively as a nutritional supplement for a large number of agricultural applications. The methods and system disclosed provide a very safe method for neutralization of UDMH with the resulting end products being non-hazardous.

In one embodiment, a chemical reaction of 1,1-Dimethylhydrazine (UDMH) with α-ketoacids is disclosed. In another embodiment, a method for neutralizing harmful effects of hydrazine comprises adding UDMH to Na2KG and adding a reducing agent to the reaction of UDMH and Na2KG. In another embodiment, a method comprises adding 1,1-Dimethylhydrazine (UDMH) to 2-ketoglutaric acid or sodium 2-ketoglutaric acid or disodium 2-ketoglutaric acid; adding a reducing agent to produce 2-(2,2-dimethylhydrazinylidene)-pentanedioic acid. It should be appreciated that other associated salts may alternatively be used. In yet another embodiment, a chemical compound comprises 2-(2,2-dimethylhydrazinylidene)-pentanedioic acid or any other associated salt.

A system for remediating areas contaminated with hydrazine can include a cleaning solution comprising a 2-ketoglutaric acid, a reducing agent for reducing the reaction of 2 KG and hydrazine, and a rinsing mechanism for rinsing equipment and/or ground surfaces contaminated with a hydrazine group compound with the cleaning solution. The system may further comprise decontaminating equipment including an aqueous solution managed by an application mechanism and comprising 2-ketoglutaric acid and an application mechanism for enabling a user in the application of the aqueous solution to equipment and/or ground surfaces accidentally contaminated with a hydrazine group compound, wherein the hydrazine group compound is converted to a stable organic compound as a result of a reaction between the aqueous solution comprising the 2-ketoglutaric acid and the hydrazine group compound and then reduced in situ by a reducing agent such as Boron.

In an exemplary embodiment, a method for neutralizing harmful effects of hydrazine comprises reacting 1,1-Dimethylhydrazine with α-ketoacids and adding a reducing agent to the reaction of 1,1-Dimethylhydrazine with the α-ketoacids. In an embodiment, the α-ketoacids comprises any salt.

In another embodiment, the α-ketoacids may comprise at least one of 2-ketoglutaric acid, sodium 2-ketoglutaric acid, and disodium 2-ketoglutaric acid.

In another embodiment, adding the reducing agent to the reaction of 1,1-Dimethylhydrazine with the α-ketoacids produces 2-(2,2-dimethylhydrazinylidene)-pentanedioic acid. The reducing agent can comprise at least one of Boron and Sodium borohydride.

In another embodiment, a chemical compound comprises 2-(2,2-dimethylhydrazinyli-dene)-pentanedioic acid formed by reacting 1,1-Dimethylhydrazine with a salt and adding a reducing agent to the reaction of 1,1-Dimethylhydrazine with the salt. In an embodiment the salt comprises at least one of α-ketoacids, 2-ketoglutaric acid, sodium 2-ketoglutaric acid, and disodium 2-ketoglutaric acid.

In an embodiment, a method for neutralizing harmful effects of hydrazine contamination comprises providing an aqueous solution comprising a 2-ketoglutaric acid, the solution adapted for application to hydrazine contaminated equipment and/or ground surfaces, applying the solution to equipment and/or ground surfaces contaminated with a hydrazine group compound, and adding a reducing agent to the reaction of the solution with the hydrazine contaminated equipment and/or ground surfaces.

In an embodiment, the hydrazine group compound is converted to a 2-(2,2-dimethylhydrazinylideneypentanedioic acid as a result of the reaction.

In another embodiment, applying the solution to equipment and/or ground surfaces contaminated with a hydrazine group compound, further comprises applying the aqueous solution to the object contaminated with the hydrazine group compound following physical removal of debris also contaminated with the hydrazine group compound from the equipment and/or ground surfaces.

In yet another embodiment, applying he aqueous solution to equipment and/or ground surfaces contaminated with a hydrazine group compound, further comprises using an application mechanism to rinse the equipment and/or ground surfaces contaminated with the hydrazine group compound with the aqueous solution.

In an embodiment, the hydrazine group compound comprises one of monomethylhdrazine (MMH) and 1,1 dimethylhydrazine (UDMH).

In an embodiment, the aqueous solution is a cleaning solution.

The embodiments and examples set forth herein are presented to best explain the present invention and its practical application and to thereby enable those skilled in the art to make and utilize the invention. Those skilled in the art, however, will recognize that the foregoing description and examples have been presented for the purpose of illustration and example only. Other variations and modifications of the present invention will be apparent to those skilled in the art, and it is the intent of the appended claims that such variations and modifications be covered. The description as set forth is not intended to be exhaustive or to limit the scope of the invention. Many modifications and variations are possible in light of the above teaching without departing from the spirit and scope of the following claims. It is contemplated that the use of the present invention can involve components having different characteristics. It is intended that the scope of the present invention be defined by the claims appended hereto, giving full cognizance to equivalents in all respects.

What is claimed is:

1. A method for neutralizing harmful effects of hydrazine said method comprising:
reacting 1,1-Dimethylhydrazine with α-ketoacids; and
adding a reducing agent to the reaction of 1,1-Dimethylhydrazine with said α-ketoacids.

2. The method of claim 1 wherein said α-ketoacids comprises any salt.

3. The method of claim 1 wherein said α-ketoacids comprise at least one of:
2-ketoglutaric acid;
sodium 2-ketoglutaric acid; and
disodium 2-ketoglutaric acid.

4. The method of claim 1 wherein adding said reducing agent to said reaction of 1,1-Dimethylhydrazine with said α-ketoacids produces 2-(2,2-dimethylhydrazinylidene)-pentanedioic acid.

5. The method of claim 1 wherein said reducing agent comprises at least one of:
Boron; and
Sodium borohydride.

6. A chemical compound comprising 2-(2,2-dimethylhydrazinylidene)-pentanedioicacid formed by reacting 1,1-Dimethylhydrazine with a salt and adding a reducing agent to the reaction of 1,1-Dimethylhydrazine with said salt.

7. The chemical compound of claim 6 wherein said salt comprises at least one of
α-ketoacids;
2-ketoglutaric acid;
sodium 2-ketoglutaric acid; and
disodium 2-ketoglutaric acid.

8. A method for neutralizing harmful effects of hydrazine contamination, said method comprising:
providing an aqueous solution comprising a 2-ketoglutaric acid, said solution adapted for application to hydrazine contaminated equipment and/or ground surfaces;
applying said solution to equipment and/or ground surfaces contaminated with a hydrazine group compound; and
adding a reducing agent to the reaction of the solution with the hydrazine contaminated equipment and/or ground surfaces.

9. The method of claim 8 wherein said hydrazine group compound is converted to a 2-(2,2-dimethylhydrazinylidene)-pentanedioic acid as a result of said reaction.

10. The method of claim 8 wherein applying said solution to equipment and/or ground surfaces contaminated with a hydrazine group compound, further comprises:
applying said aqueous solution to said object contaminated with said hydrazine group compound following physical removal of debris also contaminated with said hydrazine group compound from said equipment and/or ground surfaces.

11. The method of claim 8 wherein applying said aqueous solution to equipment and/or ground surfaces contaminated with a hydrazine group compound, further comprises:
using an application mechanism to rinse said equipment and/or ground surfaces contaminated with said hydrazine group compound with said aqueous solution.

12. The method of claim 8 wherein said hydrazine group compound comprises monomethylhdrazine (MMH).

13. The method of claim 8 wherein said hydrazine group compound comprises 1,1 dimethylhydrazine (UDMH).

14. The method of claim 1 wherein said aqueous solution is a cleaning solution.
